(19) United States
(12) Patent Application Publication
(10) Pub. No.: US 2017/0281135 A1
(43) Pub. Date: Oct. 5, 2017

(54) Image Registration Fiducials

(71) Applicant: Analogic Corporation, Peabody, MA (US)

(72) Inventors: David L. LEONG, Seabrook, NH (US); Nicholas A. ACCOMANDO, Hingham, MA (US)

(21) Appl. No.: 15/510,275

(22) PCT Filed: Sep. 12, 2014
(86) PCT No.: PCT/US2014/055325

§ 371 Date: Mar. 10, 2017

(51) Int. Cl. A61B 8/08; G06T 7/33; G06T 3/00

(52) U.S. Cl. CPC A61B 8/5238; G06T 3/0068; G06T 7/33; G06T 2207/10132

(57) ABSTRACT

A method includes receiving an input signal indicative of a set of coordinates for tissue of interest in first imaging data. The set of coordinates corresponds to user identified measurement points on a perimeter of the tissue of interest in the first imaging data. The method further includes determining a measurement for the set of measurement points. The method further includes generating a set of fiducial markers for the first imaging data corresponding to the coordinates. The method further includes registering the first imaging data with second imaging data based on the fiducial markers. The method further includes visually displaying the second imaging data with the registered first imaging data superimposed there over.

### TECHNICAL FIELD

The following generally relates to image processing and more particularly to registering images, and is described with particular application to registering ultrasound imaging data with other imaging data.

### BACKGROUND

An ultrasound (US) imaging system includes a transducer array that transmits an ultrasound beam into an examination field of view. As the beam traverses structure (e.g., of a sub-region of an object or subject) in the field of view, sub-portions of the beam are attenuated, scattered, and/or reflected off the structure, with some of the reflections (echoes) traversing back towards the transducer array. The transducer array receives echoes, which are processed to generate an image of the sub-portion of the object or subject. The image is visually displayed.

Ultrasound has been used in a wide range of medical and non-medical applications. Examples of such procedures include surgery, biopsy, therapy, etc. Such procedures have included performing scans (e.g., US and/or other) and registering imaging data generated thereby with other imaging data. In one instance, the registration includes registering pre-procedure reference imaging data with imaging data acquired at the start of or during the procedure. The later acquired imaging data would indicate changes in tissue that have occurred since the pre-procedure acquisition or last update, movement of an instrument during a procedure, etc.

The process of registering imaging data has been performed using an algorithm that includes constraints on the alignment. These constraints have typically occurred when outlining organ boundaries (image segmentation) either manually or automatically. In the case of manual boundary contouring, a qualified user draws a line around the boundary of an organ of interest. Unfortunately, this can be a time consuming process. When automated or semi-automated methods are used, the qualified user must verify the accuracy of the segmentation and accept, modify or reject it. While requiring less time than a manual segmentation, user interaction is still required, consuming time that could otherwise be spent with a patient, etc.

### SUMMARY

Aspects of the application address the above matters, and others.

In one aspect, a method includes receiving an input signal indicative of a set of coordinates for tissue of interest in first imaging data. The set of coordinates corresponds to user identified measurement points on a perimeter of the tissue of interest in the first imaging data. The method further includes determining a measurement for the set of measurement points. The method further includes generating a set of fiducial markers for the first imaging data corresponding to the coordinates. The method further includes registering the first imaging data with second imaging data based on the fiducial markers. The method further includes visually displaying the second imaging data with the registered first imaging data superimposed there over.

In another aspect, a computing apparatus includes a memory with computer executable instructions and a processor configured to execute the computer executable instructions. The processor, in response to executing the computer executable instructions: identifies a set of tissue fiducial markers in first imaging data based on a set of measurement points for anatomical tissue of interest in the first imaging data, and registers the first imaging data with second imaging data based on the set of tissue fiducial markers.

In another aspect, a computer readable storage medium is encoded with computer executable instructions, which, when executed by a processor, causes the processor to: acquire an image of a tissue of interest, identify a plurality of pairs of points on the tissue of interest for a plurality of distances measurement, one between each pair of the plurality of pairs of points, identify a set of fiducial markers from at least two pairs of the plurality of pairs of points, acquire a reference volume of imaging data, and register the image with the volume, utilizing the set of fiducial markers as constraints on the registration.

Those skilled in the art will recognize still other aspects of the present application upon reading and understanding the attached description.

### BRIEF DESCRIPTION OF THE DRAWINGS

The application is illustrated by way of example and not limitation in the figures of the accompanying drawings, in which like references indicate similar elements and in which:

FIG. 1 schematically illustrates an example system with a computing apparatus that includes computer executable instructions for automatically determining a set of fiducial markers from a set of measurement points;

FIG. 2 schematically illustrates an example of the one or more scanners;

FIG. 3 schematically illustrates an example method for registering two data sets using the set of fiducial; and

FIG. 4 schematically illustrates an example method for registering two data sets using the set of fiducial and boundary points identified in the one of the data sets.

### DETAILED DESCRIPTION

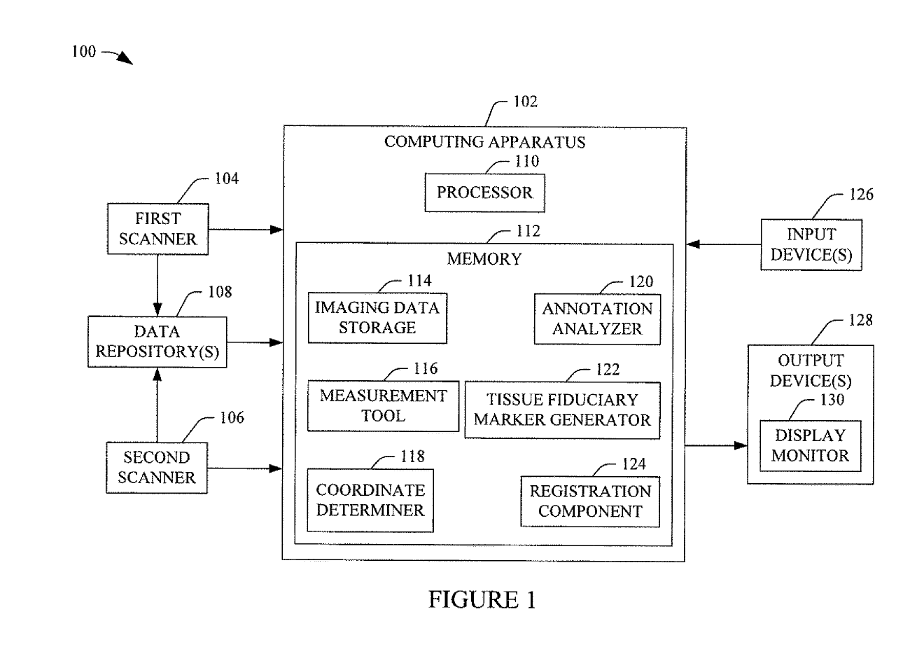

FIG. 1 illustrates a system 100 including a computing apparatus 102, a first scanner 104, a second scanner 106 and a data repository 108.

The computing apparatus 102 includes at least one processor 110 such as a microprocessor, a central processing unit, etc. The computing apparatus 102 further includes computer readable storage medium (“memory”) 112, which excludes transitory medium and includes physical memory and/or other non-transitory medium. The memory 112 stores data and/or computer executable instructions. The at least one processor 110 is configured to execute computer executable instructions, such as those in the memory 112.

In the illustrated example, the memory 112 includes imaging data storage 114 and the following modules: a measurement tool 116; a coordinate determiner 118; an annotation analyzer 120; a tissue fiduciary marker generator 122, and a registration component 124. In other embodiments, the memory 112 may include more or less modules.

The imaging data storage 114 is configured to store electronically formatted imaging data. This includes imaging data to be processed by the computing apparatus 102, imaging data being processed by the computing apparatus 102, and/or processed imaging data by the computing apparatus 102. Such imaging data can be from the first scanner 104, the second scanner 106, the data repository 108, and/or other source.

The measurement tool 116 allows for automatic, semiautomatic, and manual measurements to be taken for tissue represented in first imaging data. Such measurements at least include a distance between two points in an image (e.g., a width, a length, and/or a height of tissue of interest). In one instance, the measurement tool 116 provides a set of graphical tools in a graphical user interface. For example, the measurement tool 116 may include a graphical tool for identifying a location, in visually displayed imaging data, of the two points for the distance measurement.

The coordinate determiner 118 determines the coordinates (e.g., [x,y], [x,z], [y,z], or [y,x,z], depending on the slice orientation or volume) of each of the two measurement points used for each measurement in the imaging data space or frame of reference. In instances in which the first imaging data has been previously annotated with measurement points, the annotation analyzer 120 analyzes the first imaging data and obtains the coordinates therefrom, e.g., from a field in the electronic file.

The tissue fiduciary marker generator 122 generates tissue fiduciary markers for the first imaging data based on the measurement point coordinates. For example, the tissue fiduciary marker generator 122 generates tissue fiduciary markers with coordinates that have the same coordinates as the measurement point coordinates. By using the measurement point coordinates, the tissue fiduciary markers can be automatically generated during the normal workflow without any additional steps for or interaction by the user.

For example, where the workflow includes having a user identify two or three measurements by identifying four to six measurement points (two or three pairs, one pair for each measurement), the user simply identifies the two or three measurement points, and the tissue fiduciary markers are automatically generated therefrom. As such, this approach reduces procedure time, relative to a configuration in which the user consumes additional time segmenting and/or confirming an automatic segmentation to create fiduciary markers.

The tissue registration component 124 registers the first imaging data with second imaging data using the generated fiduciary markers. A non-limiting example of a suitable registration is described in international application serial number PCT/US13/72154, filed on Nov. 27, 2013, and entitled “Multi-Imaging Modality Navigation System,” the entirety of which is incorporated herein by reference. PCT/US13/72154 describes an approach in which a location and a spatial orientation of a 2D ultrasound slice is located and/or mapped to a corresponding plane in a 3D volume. Other registration approaches are also contemplated herein.

The computing apparatus 102 further includes an input device(s) 126 such as a mouse, keyboard, etc., and an output device(s) 128 such as a display monitor 130, a filmer, portable storage, etc. In one instance, the first imaging data is displayed via the display 130 monitor, and the input device 126 is used (e.g., by a user) to activate the measurement tool 116 and identify the measurement points, which invokes the coordinate determiner 118 to determine the coordinates and the tissue fiduciary marker generator 122 to generate the tissue fiduciary markers.

In a variation, the processor 110 and memory 112 are part of the first scanner 104, the second scanner 106, a different scanner or distributed across two or more of the first scanner 104, the second scanner 106, and different scanner. In another variation, the annotation analyzer 120 is omitted. In yet another variation, the measurement tool 116 and the coordinate determiner 118 are omitted, for example, where the imaging data has already been annotated with measurement points. In still another variation, the tissue fiduciary marker generator 122 and the registration component 124 are part of two different computing apparatuses.

The first scanner 104 and the second scanner 106 respectively generate the first and second imaging data. The first scanner 104 and the second scanner 106 can be the same imaging modality or a different imaging modality. Examples of modalities include, ultrasound (US), magnetic resonance (MR), computed tomography (CT), single photon emission computed tomography (SPECT), positron emission tomography (PET), X-ray, and/or other imaging modalities. The first and second scanners 104 and 106 can provide the first and second imaging data to the computing apparatus.

The data repository 108 stores imaging data and/or other data. In the illustrated example, this includes storing the first and/or second imaging data generated by the first and/or second scanners 104 and 106 and/or storing imaging data from the imaging data storage 114. The data repository 108 can make the first and/or second imaging data accessible to the computing apparatus 102. Examples of data repositories include a picture archiving and communication system (PACS), a radiology information system (RIS), a hospital information system (HIS), an electronic medical record (EMR), etc.

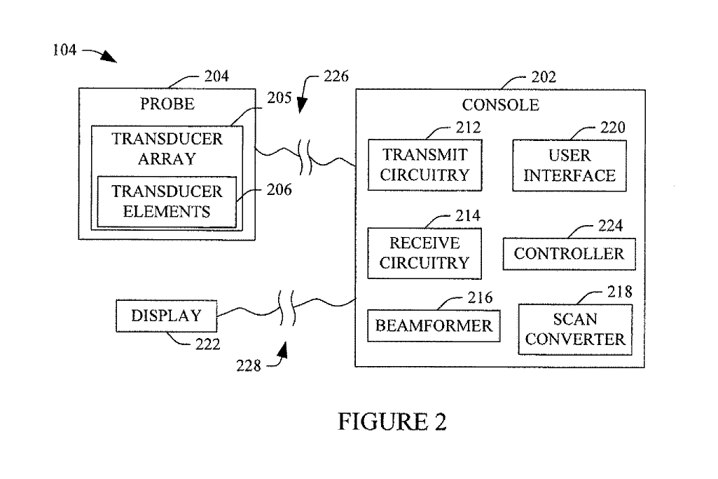

FIG. 2 illustrates an example in which the first scanner 104 includes an ultrasound (US) imaging system with a console 202 and a transducer probe 204 interfaced therewith. The transducer probe 204 includes a transducer array with a plurality of transducer elements 206. The transducer array 205 can be linear, curved, and/or otherwise shaped, fully populated, sparse and/or a combination thereof, etc. The transducer elements 206 transmit ultrasound signals and receive echo signals.

Transmit circuitry 212 selectively actuates or excites one or more of the transducer elements 206, e.g., through a set of pulses (or a pulsed signal) that is conveyed to the transducer elements 206. Receive circuitry 214 receives a set of echoes (or echo signals) generated in response to the transmitted ultrasound signals interacting with structure. The receive circuit 214 may be configured for spatial compounding, filtering (e.g., FIR and/or IIR), and/or other echo processing.

A beamformer 216 processes the received echoes. In B-mode, this includes applying time delays and weights to the echoes and summing the delayed and weighted echoes. A scan converter 218 scan converts the data for display, e.g., by converting the beamformed data to the coordinate system of a display or display region used to visually present the resulting data. A user interface (UI) 220 includes one or more input devices and/or one or more output devices for interaction between a user and the scanner 104.

A display 222 visually displays the US imaging data. A controller 224 controls the various components of the system 100. For example, such control may include actuating or exciting individual or groups of transducer elements of the transducer array 205 for an A-mode, B-mode, C-plane, and/or other data acquisition mode, steering and/or focusing the transmitted signal, etc., actuating the transducer elements 206 for steering and/or focusing the received echoes, etc.

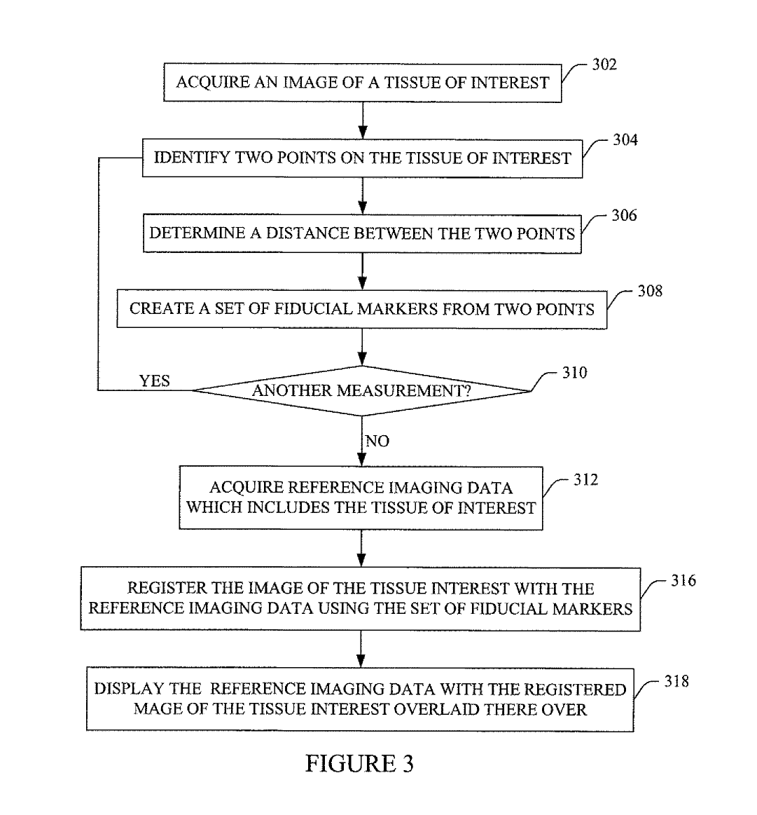

FIG. 3 illustrates a method for identifying tissue fiducial markers in imaging data and registering the imaging data with other imaging data based on the tissue fiducial markers.

It is to be appreciated that the order of the following acts is provided for explanatory purposes and is not limiting. As such, one or more of the following acts may occur in a different order. Furthermore, one or more of the following acts may be omitted and/or one or more additional acts may be added.

At 302, an image of a tissue of interest is acquired.

At 304, two points are identified on the tissue of interest.

At 306, a distance between the two points is determined.

At 308, a set of fiducial markers are created from the two points.

At 310, it is determined whether another measurement is to be taken.

If so, then at acts 304 to 310 repeated. Where the distances include a width, height and/or length of an organ, the distances can be measured in the axial and sagittal or other combination of slice planes (e.g., coronal, oblique, etc.).

Otherwise, at 312, at least one of a reference 2D image or a 3D volume (i.e., other imaging data), which includes the tissue of interest, is acquired.

At 314, the image of the region of interest is registered with the 2D image or a 3D volume using the fiducial markers.

At 316, the reference 2D image or a 3D volume is displayed with the registered image of the region of interest overlaid there over. Other examples of overlays include, but are not limited to graphics such as lines, circles, or other shapes. An overlay is not limited to image overlaid with another image.

The above may be implemented by way of computer readable instructions, encoded or embedded on computer readable storage medium, which, when executed by a computer processor(s), cause the processor(s) to carry out the described acts. Additionally or alternatively, at least one of the computer readable instructions is carried by a signal, carrier wave or other transitory medium.

The following illustrates an example of the method FIG. 3. The standard clinical workflow for a Urologist performing a biopsy of the prostate is to first measure a size of the prostate. The measurement of the prostate is performed, e.g., on an ultrasound scan by first finding a widest section of the prostate in a transverse or axial plane. The Urologist then marks the widest section of the prostate in width and height. Next the Urologist finds the centerline of the prostate in the sagittal plane and marks a depth of the prostate at its longest point. These six points on the perimeter of the prostate are automatically identified and set as fiducial markers as described herein and can be used to constrain a registration of the ultrasound image with other imaging data.

For example, these six points on the perimeter can be used to constrain a registration of the ultrasound image with a reference 2D image and/or 3D volume. The registration can be achieved by performing organ segmentation on the 2D image and/or 3D volume and locating the fiducials therein, utilizing the automated key location and matching techniques described in PCT/US13/72154, and/or otherwise. The registered data can be displayed and used for tracking and guidance during the biopsy procedure. In this example, part of the normal clinical workflow (identifying measurement points) is used to identify fiducial points that can be used as registration constraints without increasing the user workload.

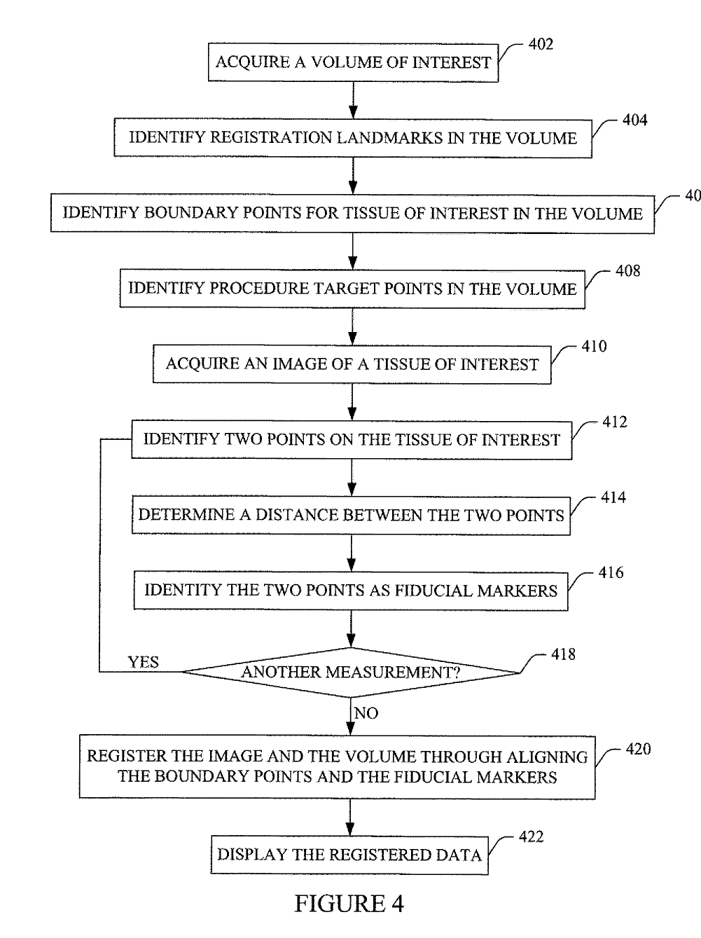

FIG. 4 illustrates another method for identifying tissue fiducial markers in imaging data and registering the imaging data with other imaging data based on the tissue fiducial markers.

It is to be appreciated that the order of the following acts is provided for explanatory purposes and is not limiting. As such, one or more of the following acts may occur in a different order. Furthermore, one or more of the following acts may be omitted and/or one or more additional acts may be added.

At 402, a volume of interest of an object or subject is scanned.

At 404, registration landmarks are identified in the volume.

At 406, boundary points for tissue of interest are identified in the volume.

At 408, procedure target points are identified in the volume.

At 410, an image of a region of interest, which includes the tissue of interest, is subsequently obtained.

At 412, two points are identified on tissue of interest in the image.

At 414, a distance between the two points is determined.

At 416, the two points are identified as fiducial markers in the image;

At 418, it is determined whether another measurement is to be taken.

If so, then at acts 412 to 418 repeated. Likewise, where the distances include a width, height and/or length of an organ, the distances can be measured in the axial and sagittal or other combination of slice planes (e.g., coronal, oblique, etc.).

Otherwise, at 420, the image of the region of interest is registered with the volume of interest based on the boundary points and the fiducial markers, for example, through aligning the boundary points and the fiducial markers.

At 422, the volume of interest is displayed with the registered image of the region of interest overlaid there over.

The above may be implemented by way of computer readable instructions, encoded or embedded on computer readable storage medium, which, when executed by a computer processor(s), cause the processor(s) to carry out the described acts. Additionally or alternatively, at least one of the computer readable instructions is carried by a signal, carrier wave or other transitory medium.

The following illustrates an example of the method FIG. 4. Volume imaging data of the prostate region of a patient is acquired. Algorithm software is used to automatically identify a cloud of keys (landmarks) distributed within the volume imaging data that are used for registration of the volume imaging data with the data of another modality, such as 2D or 3D ultrasound, in real time, as described in PCT/US13/72154. At the same time, algorithm software locates the boundary points on a perimeter of prostate. Potential lesions are also marked on the volume imaging data as target locations for biopsy sampling.

At the start of the procedure, an ultrasound sweep is be performed by the urologist, and the prostate size measured, with the measurement points identifying, e.g., six fiducial locations in the ultrasound image. The fiducials and the boundary points are compared and used to identify an initial location and orientation of the ultrasound data relative to the volume imaging data, allowing initial registration such as that described in PCT/US13/72154. The ultrasound data allows registration, tracking and guidance to be performed in real-time during the procedure for actual biopsy real-time scans. When a target potential lesion site is reached, a targeting algorithm determines where and when to take the sample. This procedure is repeated at each sample location

The application has been described with reference to various embodiments. Modifications and alterations will occur to others upon reading the application. It is intended that the invention be construed as including all such modifications and alterations, including insofar as they come within the scope of the appended claims and the equivalents thereof.

1. A method, comprising:
receiving an input signal indicative of a set of coordinates for tissue of interest in first imaging data, wherein the set of coordinates corresponds to user identified measurement points on a perimeter of the tissue of interest in the first imaging data;
determining a measurement for the set of measurement points;
generating a set of fiducial markers for the first imaging data corresponding to the coordinates;
registering the first imaging data with second imaging data based on the fiducial markers; and
visually displaying the second imaging data with the registered first imaging data superimposed there over.

2. The method of claim 1, further comprising:
receiving, prior to the registering of the first and second imaging data, a second input signal indicative of a set boundary points for the tissue of interest in the second imaging data; and
registering the first imaging data with second imaging data through aligning the set of fiducial markers in the first imaging data and the set of boundary points for the tissue of interest in the second imaging data.

3. The method of claim 2, further comprising:
receiving, prior to the registering of the first and second imaging data, a third input signal indicative of a set of user identified registration landmarks in the second imaging data; and
registering the first imaging data with second imaging data based on the set of fiducial markers in the first imaging data, the set of boundary points for the tissue of interest in the second imaging data, and the set of user identified registration landmarks in the second imaging data.

4. The method of claim 3, further comprising:
receiving, prior to the registering of the first and second imaging data, a fourth input signal indicative of target tissue of interest in the second imaging data.

5. The method of claim 3, further comprising:
registering subsequently acquired current imaging data with the second imaging data based on the set of user identified registration landmarks.

6. The method of claim 5, further comprising:
guiding an instrument to the target tissue based on the registration of the subsequently acquired current imaging data with the second imaging data.

7. The method of claim 1, wherein the first imaging data is acquired at a first time and the second imaging data is acquired at a second time, and the first time is after the second time.

8. The method of claim 1, wherein the first imaging data includes one of 2D or 3D imaging data.

9. The method of claim 1, wherein the first imaging data includes ultrasound imaging data.

10. The method of claim 1, wherein the second imaging data includes one of 2D or 3D imaging data.

11. The method of claim 1, wherein set of measurement points includes two points, and the measurement includes a distance between the two points.

12. The method of claim 1, wherein the measurement includes one of height, a width or a depth of an anatomical organ represented in the first imaging data.

13. The method of claim 12, wherein the set of measurement points include multiple pairs of points, each pair corresponding to a different measurement, and the multiple different measurements include two or more of the height, the width or the depth.

14. The method of claim 1, wherein the set of fiducial markers constrain the registration.

15. A computing apparatus, comprising:
a memory with computer executable instructions; and
a processor configured to execute the computer executable instructions, wherein the processor, in response to executing the computer executable instructions:
identifies a set of tissue fiducial markers in first imaging data based on a set measurement points for anatomical tissue of interest in the first imaging data; and
registers the first imaging data with second imaging data based on the a set of tissue fiducial markers.

16. The computing apparatus of claim 15, wherein the first imaging data is included in an electronically formatted file, and the processor, in response to executing the computer executable instructions, further: retrieves the set measurement points from the electronically formatted file, and identifies the set of tissue fiducial markers based on the retrieved set of measurement points.

17. The computing apparatus of claim 15, wherein the processor, in response to executing the computer executable instructions, further: receives a user input indicative of the set measurement points, and identifies the set of tissue fiducial markers based on the user input.

18. The computing apparatus of claim 15, wherein the second imaging data includes of a set points on a perimeter of the tissue of interest, and the processor, in response to executing the computer executable instructions, further: registers the first imaging data with second imaging data based on the set of tissue fiducial markers and the set points on a perimeter of the tissue of interest.

19. The computing apparatus of claim 18, wherein the second imaging data includes of a set of regions of interest, and the processor, in response to executing the computer executable instructions, further: registers the first imaging data with second imaging data based on the set of tissue fiducial markers, the set points on a perimeter of the tissue of interest, and the set of regions of interest.

20. The computing apparatus of claim 15, wherein the first imaging data includes one of 2D or 3D imaging data.

21. The computing apparatus of claim 20, wherein the first imaging data includes ultrasound imaging data.

22. The computing apparatus of claim 15, wherein set of measurement points includes two points, and the measurement includes a distance between the two points.

23. The computing apparatus of claim 15, wherein the measurement includes one of height, a width or a depth of an anatomical organ represented in the first imaging data.

24. The computing apparatus of claim 23, wherein the set of measurement points include multiple pairs of points, each pair corresponding to a different measurement, and the multiple different measurements include two or more of the height, the width or the depth.

25. A computer readable storage medium encoded with computer executable instructions, which, when executed by a processor, causes the processor to:
acquire an image of a tissue of interest;
identify a plurality of pairs of points on the tissue of interest for a plurality of distances measurement, one between each pair of the plurality of pairs of points;
identify a set of fiducial markers from at least two pairs of the plurality of pairs of points;
acquire a reference volume of imaging data; and
register the image with the volume, utilizing the set of fiducial markers as constraints on the registration.

26. A computer readable storage medium encoded with computer executable instructions, which, when executed by a processor, causes the processor to:
identify, prior to registering image with the volume, boundary points on the tissue of interest in the volume; and
register the image with the volume based on the set of fiducial markers and the boundary points.
